(19) United States
(12) Patent Application Publication
(10) Pub. No.: US 2008/0175895 A1
(43) Pub. Date: Jul. 24, 2008

(54) SYSTEM, DEVICES, AND METHODS FOR IONTOPHORETIC DELIVERY OF COMPOSITIONS INCLUDING ANTIOXIDANTS ENCAPSULATED IN LIPOSOMES

(76) Inventors: Kentaro Kogure, Sapporo (JP); Moeko Miyashita, Sapporo (JP); Hideyoshi Harashima, Sapporo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/962,011

(22) Filed: Dec. 20, 2007

Related U.S. Application Data
(60) Provisional application No. 60/886,762, filed on Jan. 26, 2007.

(30) Foreign Application Priority Data
Jan. 16, 2007 (JP) 2007-007130

(51) Int. Cl. A61K 9/127; A61K 38/44; A61P 25/28; A61P 25/16; A61P 35/00; A61P 17/00

(52) U.S. Cl. 424/450; 424/94.4

(57) ABSTRACT

Systems, devices, and methods for delivering one or more active ingredients to intradermal tissues, deep regions of pores, and intradermal tissues in the vicinity of the pores. In some embodiments, a composition is provided including a plurality of liposomes including a cationic lipid, and an amphiphilic glycerophospholipid having a saturated fatty acid moiety and an unsaturated fatty acid moiety, and one or more antioxidants and/or antioxidant enzymes.

### CROSS-REFERENCES TO RELATED APPLICATIONS

This application claims benefit of U.S. Provisional Application No. 60/886,762 filed Jan. 26, 2007, the entire contents of which are incorporated herein by reference.

### BACKGROUND

1. Technical Field

This disclosure generally relates to the field of transdermal administration of active ingredients by iontophoresis and, more particularly, to compositions useful for transdermally administering an antioxidant via iontophoresis to, for example, deep regions of a pore and intradermal tissue around the pore.

2. Description of the Related Art

Chronic repeated exposure to ultraviolet radiation (e.g., solar ultraviolet radiation) and free-radicals (e.g., reactive oxygen species such as, for example, hydroxyl radicals, hydrogen peroxides, singlet oxygens, and superoxide ions) may induce acute and chronic reactions in the skin, eye, and immune system of animals and humans. Although the sun emits ultraviolet radiation in the Ultraviolet A, B, and C bands, most of the ultraviolet radiation that reaches the Earth's surface is Ultraviolet A (UVA). UVA penetrates much deeper into the skin than any of the other ultraviolet wavelengths. Prolonged exposure to ultraviolet radiation may cause immunosuppression, photoaging, cellular damage, and skin damage (e.g., skin cancers, skin tumor development, actinic keratosis, and malignant melanoma). Such dermatopathy is known to occur not only on the surface of a skin, but also within the skin. Accordingly, there is a need for treatments that are capable of inhibiting the production of reactive oxygen species and/or capable of quenching reactive oxygen species found within the skin. There is also a need for treatments that are capable of converting free radical oxygen species to a compound that is less toxic to the body and that may ultimately be metabolized.

Although skin is one of the most extensive and readily accessible organs, it has historically been difficult to deliver certain active ingredients transdermally. Often a drug is administered to a living body mainly through the corneum of the skin. The corneum, however, is a lipid-soluble high-density layer that makes the transdermal administration of high water-soluble substances and polymers such as peptides, nucleic acids, and the like difficult.

Iontophoresis employs an electromotive force and/or current to transfer an active ingredient (e.g., a charged substance, an ionized compound, an ionic a drug, a therapeutic, a bioactive-agent, and the like), to a biological interface (e.g., skin, mucus membrane, and the like), by applying an electrical potential to an electrode proximate an iontophoretic chamber comprising a similarly charged active ingredient and/or its vehicle. For example, a positively charged ion is transferred into the skin at an anode side of an electric system of an iontophoresis device. In contrast, a negatively charged ion is transferred into the skin at a cathode side of the electric system of the iontophoresis device.

Commercial acceptance of transdermal delivery devices or pharmaceutically acceptable carriers is dependent on a variety of factors including cost to manufacture, shelf life, stability during storage, efficiency and/or timeliness of active ingredient delivery, biological capability, and/or disposal issues. Commercial acceptance of transdermal delivery devices or pharmaceutically acceptable carriers is also dependent on their versatility and ease-of-use.

The present disclosure is directed to overcoming one or more of the shortcomings set forth above, and/or providing further related advantages.

### BRIEF SUMMARY

Oxidative stress generally refers to the steady state level of oxidative damage in a cell, tissue, or organ caused by reactive oxygen species. Oxidative stress results in part from an imbalance between the rate at which oxidative damage (caused by reactive oxygen species) is induced and the rate at which the damage is efficiently repaired and removed. The rate at which damage is caused is determined by how fast the reactive oxygen species are generated and then inactivated by, for example, defense agents (e.g., antioxidants).

Most life forms maintain a reducing environment within their cells. This reducing environment is preserved by enzymes that maintain the reduced state through a constant input of metabolic energy. Disturbances in this normal redox state can cause toxic effects through the production of, for example, peroxides and free radicals that damage components of the cell, including proteins, lipids, and DNA.

In humans, oxidative stress is involved in many diseases, such as atherosclerosis, Parkinson's disease, and Alzheimer's disease, as well as in many important biological process including those involved in the prevention of ageing, the killing of pathogens, and cell signaling.

In one aspect, the present disclosure is directed to a composition for iontophoretic delivery of one or more antioxidant enzymes. The composition comprises a plurality of liposomes and one or more antioxidant enzymes being carried (e.g., encapsulated, and the like) by the liposomes. In some embodiments, the pluralities of liposomes include a cationic lipid, and an amphiphilic glycerophospholipid having a saturated fatty acid moiety and an unsaturated fatty acid moiety. In some embodiments, the one or more antioxidant enzymes are selected from the group consisting of superoxide dismutase (SOD), glutathione peroxydase (GSH-Px), and catalase.

In another aspect, the present disclosure is directed to a method for treating a condition or a disease associated with oxidative stress in a living biological subject. The method includes iontophoretically administering to the living biological subject a composition comprising a plurality of liposomes and one or more antioxidant enzymes being carried by the plurality of liposomes. In some embodiments, the pluralities of liposomes include a cationic lipid, and an amphiphilic glycerophospholipid having a saturated fatty acid moiety and an unsaturated fatty acid moiety. In some embodiments, the one or more antioxidant enzymes are selected from the group consisting of superoxide dismutase (SOD), glutathione peroxydase (GSH-Px), and catalase. In some embodiments, the cationic lipid is present in a molar ratio of the cationic lipid to the amphiphilic glycerophospholipid of about 3:7 to about 7:3. In some embodiments, the liposomes have an average particle diameter ranging from about 400 to about 1000 nm.

The method may further include providing a sufficient amount of current to deliver a therapeutic effective amount of the composition to the living biological subject.

In another aspect, the present disclosure is directed to a method for preventing oxidative damage in a biological subject. The method includes iontophoretically administering to the biological subject in need of such treatment a therapeutically effective amount of a composition comprising a plurality of liposomes comprising a cationic lipid, an amphiphilic glycerophospholipid having a saturated fatty acid moiety and an unsaturated fatty acid moiety, and one or more antioxidant enzymes selected from the group consisting of superoxide dismutase (SOD), glutathione peroxydase (GSH-Px), and catalase. In some embodiments, the cationic lipid is present in a molar ratio of the cationic lipid to the amphiphilic glycerophospholipid of about 3:7 to about 7:3.

In another aspect, the present disclosure is directed to a formulation that enables the stable, efficient, iontophoretic delivery of an antioxidant component to each of the deep regions of a pore and intradermal tissue around the pore. In some embodiments, an antioxidant component has been encapsulated in a liposome to provide stable, efficient delivery of the antioxidant component to each of the deep regions of a pore and intradermal tissue around the pore. In some embodiments, the disclosed methods include compositions and/or formulations that provide for the administering an antioxidant component to a living organism by iontophoresis and are characterized in that the antioxidant component is encapsulated in a liposome. In some embodiments, the antioxidant component is an antioxidant enzyme. In some embodiments, the antioxidant enzyme is superoxide dismutase (SOD).

In some embodiments, the antioxidant enzyme may be glutathione peroxidase (GSD-Px) and/or catalase. In addition, the liposome formulation may comprise a combination of liposomes in each of which superoxide dismutase (SOD), glutathione peroxidase (GSH-Px), or catalase is encapsulated as the antioxidant component. In some embodiments, the liposome contains, as constituents, a cationic lipid, and an amphiphilic glycerophospholipid containing both a saturated fatty acid moiety and an unsaturated fatty acid moiety as constituent fatty acids.

In another aspect, the present disclosure is directed to a liposome formulation useful for the prevention or therapy of a dermatopathy resulting from ultraviolet light.

In some embodiments, an electrode assembly for administering an antioxidant component to a living organism by iontophoresis is provided. The iontophoresis device includes an electrode assembly and is operable to iontophoretically deliver any of the disclosed compositions and/or formulations.

In some embodiments, the disclosed compositions and/or formulations include antioxidant encapsulated liposomes that are stable and suitable for delivery to a skin pore. In some embodiments, the disclosed compositions and/or formulations can be delivered intradermally via iontophoresis. Accordingly, in some embodiments, active oxygen produced in the skin can be extinguished. In some embodiments, the disclosed compositions and/or formulations may be useful for the prevention, reduction, and/or therapy of a dermatopathy resulting from, for example, irradiation with ultraviolet light. In some embodiments, the disclosed compositions and/or formulations may be useful for treating injuries resulting from the generation of active oxygen in skin, which have traditionally been considered difficult to treat or prevent. In some embodiments, the disclosed compositions and/or formulations may be useful for the prevention of a dermatopathy including skin inflammation, as well as the suppression of the generation of spots and wrinkles resulting from oxidative stress damage.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is a schematic diagram of an iontophoresis device used in an in vivo skin penetration test upon administration of a liposome formulation according to one illustrated embodiment.

FIG. 2A is a photograph showing the biological morphology of the skin of a rat that was irradiated with ultraviolet light without the iontophoretic administration of superoxide dismutase (SOD)-carrying liposome formulation according to one illustrated embodiment.

FIG. 2B is a photograph showing the biological morphology of skin from a rat that was irradiated with ultraviolet light after the administration of the SOD-carrying liposome formulation according to one illustrated embodiment.

FIG. 3 is a bar plot showing the results of a determination of the amount of a lipid peroxide (malon dialdehyde: MDA) from skin from a rat to which a SOD-carrying liposome formulation is not administered (UV) and skin from a rat to which a SOD-carrying liposome formulation is administered (SOD) after irradiation with UV, according to multiple illustrated embodiments.

FIG. 4A is a micrograph of a skin section of a rat to which the SOD-carrying liposome formulation was not iontophoretically administered (marked as UV) and that was subjected to immunostaining with anti-(hexanoyl)lysine (HEL) according to one illustrated embodiment.

FIG. 4B is a micrograph of a skin section of a rat to which the SOD-carrying liposome formulation was not iontophoretically administered (UV) and that was subjected to immunostaining with anti-malon dialdehyde (MDA) according to one illustrated embodiment.

FIG. 4C is a micrograph of a skin section of a rat to which the SOD-carrying liposome formulation was not iontophoretically administered (UV) and that was subjected to immunostaining with anti-8-OH-deoxyguanosine (8-OHdG) according to one illustrated embodiment.

FIG. 4D is a micrograph of a skin section of a rat to which the SOD-carrying liposome formulation was iontophoretically administered (SOD) and that was subjected to immunostaining with anti-(hexanoyl)lysine (HEL) according to one illustrated embodiment.

FIG. 4E is a micrograph of a skin section of a rat to which the SOD-carrying liposome formulation was iontophoretically administered (SOD) and that was subjected to immunostaining with anti-malon dialdehyde (MDA) according to one illustrated embodiment.

FIG. 4F is a micrograph of a skin section of a rat to which the SOD-carrying liposome formulation was iontophoretically administered (SOD) and that was subjected to immunostaining with anti-8-OH-deoxyguanosine (8-OHdG) according to one illustrated embodiment.

FIG. 5 is a flow diagram of a method for treating a condition or a disease associated with oxidative stress in a living biological subject according to one illustrated embodiment.

FIG. 6 is a flow diagram of a method for preventing oxidative damage in a biological subject according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with electrically powered devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment,” or “in another embodiment,” or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoretic delivery liposome formulation, including an “antioxidant” includes a single antioxidant, or two or more antioxidants. It should also be noted that the term “or” is generally employed in its sense including “and/or” unless the content clearly dictates otherwise.

Unless otherwise specified, the variable “Cₙ” in a group, or as part of a group, generally refers to the “total number of carbon atoms n” in the group or the part of a group. Thus, for example, “C₁₋₆ saturated fatty acid” refers to a “saturated fatty acid containing from 1 to 6 carbon atoms”, and “C₁₂₋₃₁ cholesteryl fatty acid ester” refers to a “cholesteryl fatty acid ester containing from 12 to 31 carbon atoms”.

The terms “alkyl”, “alkenyl”, or “alkynyl” as a group or as part of a group generally refer to, unless otherwise specified, straight chain, branched chain, cyclic, substituted, or unsubstituted hydrocarbon radicals. In some embodiments, the “alkyl”, “alkenyl”, or “alkynyl” are selected from the group consisting of straight chain alkyls, alkenyls, or alkynyls and branched chain alkyls, alkenyls, or alkynyls. In some embodiments, the “alkyl”, “alkenyl”, or “alkynyl” is selected from the group consisting of straight chain alkyls, alkenyls, and alkynyls.

The term “aryl” generally refers to, unless otherwise specified, aromatic monocyclic or multicyclic hydrocarbon ring system consisting only of hydrogen and carbon and containing from 6 to 19 carbon atoms, where the ring system may be partially or fully saturated. Aryl groups include, but are not limited to, groups such as phenyl and naphthyl.

The term “heteroaryl” generally refers to, unless otherwise specified, a 5- to 6-membered partially or fully aromatic ring radical which consists of one to three heteroatoms selected from the group consisting of nitrogen, oxygen, and sulfur.

The term “front surface” generally refers, unless otherwise specified, to a side near the skin of a living body on the path of electric current flowing through the inside of the electrode structure in administering liposomes.

The term “antioxidant component,” or “antioxidant,” or “antioxidant enzyme” generally refers to, unless otherwise specified, a compound, molecule, substance, or treatment capable of reducing oxidative damage caused by, for example, free radicals (e.g., reactive oxygen species such as, for example, hydroxyl radicals, hydrogen peroxides, singlet oxygens, and superoxide ions).

Among “antioxidant components” examples include “antioxidants” and “antioxidant enzymes.”

Among antioxidants examples include fat-soluble antioxidants such as, for example, α-tocopherol (vitamin E), β-carotene, astaxanthin, lycopene, capsaicin, and the like. Further examples of antioxidants include water-soluble antioxidants such as, for example, ascorbic acid (vitamin C), polyphenol antioxidants such as curcumin, cysteine, and the like.

Among antioxidant enzymes examples include catalasey, glutathione peroxidases (e.g., Peroxidase (GSH-Px)), glutathione-S-transferase (GST), derum paraoxonase (PON), superoxide dismutase (SOD), and the like.

Iontophoretic delivery of active ingredients (e.g., antioxidants, antioxidant enzymes, and the like) may provide a way of avoiding the first-pass effect of the liver, and may permit for easier control of initiation, cessation, etc., associated with the administration of a drug.

Although it may be possible to transdermally administer substances with various physico-chemical properties using charged liposomes as a carriers (see e.g., MedianVM et al., International Journal of Pharmaceutics, Dec. 8, 2005:306(1-2):1-14. Epub Nov. 2, 2005 Epub Nov. 2, 2005), the large particle diameter of liposomes, often make it difficult to pass through the corneum.

Hair follicles, which are connected from the skin surface to a deep region of the skin, may provide a route of transdermally administering liposomes efficiently (e.g., Hoffman R T et al., Nat Med. July 1995; 1(7):705-706; Fleisher D et al, Life Sci. 1995;57 (13):1293-1297). It may be possible to, for example, administer liposomes enclosing an enzyme to hair follicle stem cells in hair follicles by iontophoresis (see e.g., Protopapa EE et al., J Eur Acad Dermatol Venereol. July 1999;13(1):28-35). It may also be possible to, for example, administer liposomes enclosing 5-aminolevulinic acid serving as an agent for a photodynamic therapy to the hair follicle sebaceous gland and the like in upper regions of hair follicles by iontophoresis (see e.g., Han I et al., Arch Dermatol Res. November 2005; 295(5):210-217. Epub Nov. 11, 2005). Han I et al. has also reported that liposomes enclosing adriamycin serving as an agent for treating hair follicle-associated tumors may be delivered to hair follicles by iontophoresis (Han I et al., Exp Dermatol. February 2004; 13(2):86-92).

Often in iontophoresis, a drug is administered to upper regions of skin tissues. In some embodiments, a drug (e.g., antioxidants, antioxidant enzymes, and the like) is systemically administered to a general circulation system through subcutaneous blood vessels present in the deep region of a skin. In some embodiments, there may exist a need to inhibit production of active oxygen or to quickly remove active oxygen species within the skin resulting from, for example, ultraviolet light exposure. In such cases, it may be desirable to reliable delivery an antioxidant component to an intradermal tissue around a pore for inhibiting active oxygen production within the skin or for quickly removing produced active oxygen species. Accordingly, some embodiments disclose stable, efficient delivery of an antioxidant component to each of the deep regions of a pore and an intradermal tissue around the pore via iontophoresis.

### Liposome Composition for Iontophoresis

As described above, in some embodiments, the disclosed composition includes an active ingredient (e.g., antioxidants, antioxidant enzymes, and the like) carried in a liposome, in which the liposome includes, as a constituent component, a cationic lipid, and an amphiphilic glycerophospholipid including both saturated fatty acid and an unsaturated fatty acid moieties. It is an unexpected fact that liposomes comprising such specific constituent components advantageously provide stable deliver of one or more antioxidant components to deep regions of a pore and/or intradermal tissues in the vicinity of the pore by iontophoresis.

In some embodiments, a composition is provided for administering an active ingredient through a pore and/or intradermal tissues in the vicinity of the pore. The composition includes a plurality of liposomes and an active ingredient carried by the liposomes. The liposomes may include a cationic lipid and an amphiphilic glycerophospholipid.

The cationic lipid may comprise a C₁₋₂₀ alkane substituted with a C₁₋₂₀ acyloxy group and a triC₁₋₄ alkylammonium group. In some embodiments, the C₁₋₂₀ alkane is a C₁₋₅ alkane. In some other embodiments, the C₁₋₂₀ alkane is a C₁₋₃ alkane. In some embodiments, the C₁₋₂₀ alkane may comprise from one to four C₁₋₂₀ acyloxy groups. In some embodiments, the C₁₋₂₀ alkane may comprise two C₁₋₂₀ acyloxy groups. In some embodiments, the C₁₋₂₂ acyloxy groups are C₁₋₂₀ acyloxy groups. In some embodiments, the C₁₋₂₂ acyloxy groups are C₁₋₁₈ acyloxy groups.

Among the C₁-C₂₂ acyloxy groups examples include an alkyl carbonyloxy group, an akenyl carbonyloxy group, an alkynyl carbonyloxy group, an aryl carbonyloxy group, or a heteroaryl carbonyloxy group. In some embodiments, the C₁-C₂₂ acyloxy group is selected from the group consisting of an alkyl carbonyloxy group, an akenyl carbonyloxy group, and an alkynyl carbonyloxy. In some embodiments, the C₁-C₂₂ acyloxy group is an akenyl carbonyloxy group.

The above-mentioned C₁₋₂₀ alkane may include, as a substituent, preferably one to four triC₁₋₆ alkylammonium groups. In some embodiments, the C₁₋₂₀ alkane may include one triC₁₋₆ alkylammonium group. In some embodiments, the triC₁₋₆ alkylammonium groups are triC₁₋₄ alkylammonium groups. In some embodiments, the triC₁₋₆ alkylammonium groups may carry one or more counter ions. Examples of counter ions of the above-mentioned trialkylammonium group include, but are not limited to, chlorine ions, bromine ions, iodine ions, fluorine ions, sulfurous ions, nitrous ions, etc. In some embodiments, the counter ion is a chlorine ion, bromine ion, or iodine ion.

Specific examples of the cationic lipid include preferably 1,2-dioleoyloxy-3-trimethylammonium propane (DOTAP), dioctadecyldimethylammonium chloride (DODAC), N-(2,3-dioleyloxy)propyl-N,N,N-trimethylammonium (DOTMA), didodecylammonium bromide (DDAB), 1,2-dimyristoyloxypropyl-3-dimethylhydroxyethylammonium (DMRIE), and 2,3-dioleyloxy-N-[2(sperminecarboxamido)ethyl]-N,N,-dimethyl-1-propanaminum trifluoroacetate (DOSPA). In some embodiments, the cationic lipid is DOTAP.

In some embodiments, the amphiphilic glycerophospholipid comprises a saturated fatty acid moiety and an unsaturated fatty acid moiety.

In some embodiments, the amphiphilic glycerophospholipid includes both a saturated fatty acid moiety and an unsaturated fatty acid moiety. In some embodiments, the amphiphilic glycerophospholipid is selected from the group consisting of phosphatidylcholine, phosphatidylethanolamine, phosphatidylglycerol, phosphatidic acid, cardiolipin, phosphatidylserine, phosphatidylinositol, and the like. In some embodiments, the amphiphilic glycerophospholipid is phosphatidylcholine. In some embodiments, the amphiphilic glycerophospholipid is an egg-yolk phosphatidylcholine.

In some embodiments, the amphiphilic glycerophospholipid includes a saturated fatty acid moiety selected from the group consisting of C₁₂₋₂₂ saturated fatty acids and C₁₄₋₁₈ saturated fatty acids. In some embodiments, the amphiphilic glycerophospholipid comprises at least one fatty acid moiety selected from the group consisting of palmitic acid, lauric acid, myristic acid, pentadecylic acid, margaric acid, stearic acid, tuberculostearic acid, arachidic acid, and behenic acid. In some embodiments, the amphiphilic glycerophospholipid comprises at least one fatty acid moiety selected from the group consisting of palmitic acid, myristic acid, pentadecylic acid, margaric acid, and stearic acid.

Among the unsaturated fatty acid moieties, examples include C₁₄₋₂₂ unsaturated fatty acids and C₁₄₋₂₀ unsaturated fatty acids. In some embodiments, the unsaturated fatty acid moiety comprises from 1 to 6 carbon-carbon double bonds. In some embodiments, the unsaturated fatty acid moiety comprises from 1 to 4 carbon-carbon double bonds.

In some embodiments, the unsaturated fatty acid includes at least one moiety selected from the group consisting of oleic acid, myristoleic acid, palmitoleic acid, elaidic acid, vaccenic acid, gadoleic acid, erucic acid, nervonic acid, linoleic acid, α-linoleic acid, eleostearic acid, stearidonic acid, arachidonic acid, eicosapentaenoic acid, clupanodonic acid, and docosahexaenoic acid. In some embodiments, the unsaturated fatty acid includes at least one moiety selected from the group consisting of oleic acid, myristoleic acid, palmitoleic acid, elaidic acid, vaccenic acid, gadoleic acid, erucic acid, nervonic acid, linoleic acid, α-linoleic acid, eleostearic acid, and stearidonic acid.

In some embodiments, the amphiphilic glycerophospholipid includes both a saturated fatty acid moiety and an unsaturated fatty acid moiety. In some embodiments, the saturated fatty acid moiety is selected from the group consisting of palmitic acid, myristic acid, pentadecylic acid, margaric acid, and stearic acid, and the unsaturated fatty acid moiety is selected from the group consisting ofoleic acid, myristoleic acid, palmitoleic acid, elaidic acid, vaccenic acid, gadoleic acid, erucic acid, nervonic acid, linoleic acid, α-linoleic acid, eleostearic acid, stearidonic acid, and arachidonic acid.

In some embodiments, the liposomes further comprise a sterol as a constituent component. The sterol may be selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether. In some embodiments, the sterol may be selected from the group consisting of cholesterol, cholesteryl lanoate, cholesteryl oleate, cholesteryl nonanoate, macadamia nut fatty acid cholesteryl, and dihydrocholesterol polyethylene glycol ether (e.g., dihydrocholes-30). In some embodiments, the sterol is cholesterol.

In some embodiments, the fatty acid moiety such as, for example, cholesteryl fatty acid, dihydrocholesteryl fatty acid, and the like may be saturated or unsaturated. In some embodiments, the fatty acid moiety may be a straight chain, branched chain, or cyclic fatty acid. In some embodiments, the fatty acid moiety in the cholesteryl fatty acid may be a straight chain fatty acid, and the fatty acid moiety in the dihydrocholesteryl fatty acid may be a straight chain fatty acid.

The liposomes may comprise an active ingredient (e.g., an antioxidant, antioxidant enzyme, and the like), a cationic lipid, and an amphiphilic glycerophospholipid. The stability and iontophoretic delivery efficiency of the liposomes may depend on the ratio of the cationic lipid to the amphiphilic glycerophospholipid present in the liposomes. In some embodiments, a molar ratio of the cationic lipid to the amphiphilic glycerophospholipid ranges from about 3:7 to about 7:3. In some embodiments, a molar ratio of the cationic lipid to the amphiphilic glycerophospholipid ranges from about 4:6 to about 6:4. In some embodiments, when the liposomes include a sterol, a molar ratio of the cationic lipid to the sterol ranges from about 3:7 to about 7:3. In some embodiments, a molar ratio of the cationic lipid to the sterol ranges from about 4:6 to about 6:4.

In some embodiments, a molar ratio of the amphiphilic glycerophospholipid to the sterol ranges from about 3:7 to about 7:3. In some embodiments, a molar ratio of the amphiphilic glycerophospholipid to the sterol ranges from about 4:6 to about 6:4. In some embodiments, a molar ratio of the cationic lipid to the total of the amphiphilic glycerophospholipid and the sterol ranges from about 3:7 to about 7:3. In some embodiments, a molar ratio of the cationic lipid to the total of the amphiphilic glycerophospholipid and the sterol ranges from about 4:6 to about 6:4. In some embodiments, a molar ratio of the cationic lipid, to the amphiphilic glycerophospholipid, and to the sterol is about 2:1:1.

In some embodiments, the average particle diameter of the liposomes is about 400 nm or greater. In some embodiments, the average particle diameter of the liposomes ranges from about 400 nm to about 1000 nm. The average particle diameter of the liposomes can be confirmed by, for example, a dynamic-light-scattering method, a static-light-scattering method, an electron microscope observation method, and an atomic force microscope observation method.

### Antioxidant Components

In some embodiments, an iontophoretic delivery composition may include one or more active ingredients in the form of a hydrophobic substance or a water soluble substance. In some embodiments, the one or more active ingredients (e.g., antioxidants, antioxidant enzymes, and the like) may comprise an ionic, cationic, ionizeable, and/or neutral substance insofar as it can be carried (e.g., encapsulated) in a liposome.

In some embodiments, the active ingredient may comprise one or more antioxidant components (e.g., antioxidants, antioxidant enzymes, and the like). In some embodiments, the antioxidant component may comprise an antioxidant enzyme such as, for example, an active oxygen-extinguishing and/or quenching enzyme. In some embodiments, the antioxidant enzyme is superoxide dismutase (SOD) as an active oxygen-extinguishing enzyme. In some embodiments, the antioxidant enzyme is glutathione peroxidase (GSH-Px) and/or catalase.

In some embodiments, a liposome formulation may be composed of a combination of liposomes in each of which at least one of a superoxide dismutase (SOD), glutathione peroxidase (GSH-Px), and/or catalase is encapsulated as an antioxidant component.

In some embodiments, the composition may include a fat-soluble antioxidant compound such as α-tocopherol (vitamin E), β-carotene, astaxanthin, lycopene, or capsaicin, or a water-soluble antioxidant compound such as ascorbic acid (vitamin C), a polyphenol antioxidants such as curcumin, or cysteine.

The disclosed liposomes and composition comprising liposomes may be prepared in a variety of ways. In some embodiments, the disclosed liposomes, compositions, and/or formulations comprising liposomes may be prepared by the following Example 1.

### EXAMPLE 1

First, cationic lipid, amphiphilic glycerophospholipid, and optionally sterol or the like are mixed in desired ratios in an organic solvent such as CHCl₃ to obtain a suspension. The suspension is distilled under reduced pressure, and the addition of an organic solvent and distillation under reduced pressure are repeated, to yield a lipid film. Next, to the lipid film, a buffer such as 10 mM to 50 mM HEPES (2-[4-(2-hydroxyethy)-1piperazinyl]ethanesulfonic acid) or the like and a desired amount of active ingredient are added. The resulting mixture is left standing at room temperature for 10 minutes for hydration, followed by sonication. The sonication is performed in a sonicator, for example, at room temperature at 85 W for 1 minute, but the conditions are not limited thereto. The mixture is treated using a membrane filter, extruder, etc., to adjust the particle diameter, thereby obtaining liposomes. The liposomes are further mixed with a pharmacologically acceptable carrier and the like, thereby obtaining a composition and/or formulation of liposomes.

A number of pharmacologically acceptable carriers and excipients may be used with the disclosed compositions and/or formulations, and methods insofar as the administration of liposomes by iontophoresis is not substantially hindered. For example, surfactants, lubricants, dispersants, buffers such as HEPES, additives such as preservatives, solubilizing agents, antiseptics, stabilizing agents, antioxidants, colorants, may be included. The liposome composition can be formed into a suitable dosage form as desired, insofar as the administration of liposomes by iontophoresis is not substantially hindered.

In some embodiments, the composition of liposomes is formed into a solution or suspension with HEPES buffer and/or any of the disclosed electrolytes. The disclosed composition and methods can be applied to various uses according to types and properties of an active ingredient to be enclosed in liposome.

### Application of Liposome Formulation

The disclosed liposome compositions and/or formulations can be advantageously utilized in, for example, the prevention or therapy of a local dermatopathy, the intradermal administration of an antioxidant component, and the therapy for the extinction of active oxygen requiring a systemic action. Accordingly, in some embodiments, the disclosed liposome compositions and/or formulations may enable the stable, efficient delivery of an antioxidant component to each of the deep regions of a pore and intradermal tissues surrounding the pore.

In some embodiments, a method of administering an antioxidant component to a living organism by iontophoresis includes placing any of the disclosed compositions and/or formulations on the skin surface of a living body, and applying an electric current to the skin. In some embodiments, the antioxidant component is carried (e.g., enclosed, encapsulated, and the like) in the liposomes in the composition and administered to a living organism through, for example, a skin pore.

In some embodiments, the disclosed compositions and/or formulations may be directly placed on the skin surface, or may be part of an electrode structure of an iontophoresis device in which the composition is held, stored, or carried. In use, electric current is applied to an electrode structure holding, storing, or carrying a composition of liposomes encapsulating an antioxidant component, and administered iontophoretically.

For cationic liposomes, the anode of an iontophoresis device is supplied with an electric current. In some embodiments, the electric current supplied by the iontophoretic device and applied to the liposomes ranges from about 0.1 mA/cm² to about 0.6 mA/cm². In some embodiments, the electric current supplied by the iontophoretic device ranges from about 0.3 mA/cm² to about 0.5 mA/cm². In some embodiments, the electric current supplied by the iontophoretic device is about 0.45 A/cm². In some embodiments, a period of time for applying electric current to the electrode structure ranges from about 0.5 hours to about 1.5 hours, in some embodiments, from about 0.75 hours to about 1.25 hours, and, in some further embodiments, about 1 hour.

In some embodiments, the living organism includes any mammal, such as, for example, a rat, a human being, a guinea pig, a rabbit, a mouse, and a pig. In some embodiments, the living organism is a human being.

### Electrode Assembly and Device for Iontophoresis

In some embodiments, the disclosed compositions and/or formulations may be held in, stored, carried, or be part of, an electrode structure suitable for iontophoretic delivery of the compositions and/or formulations. In some embodiments, the electrode structure for administering an active ingredient (e.g., antioxidant component, and the like) to a living body via iontophoresis comprises one or more of the disclosed compositions and/or formulations. In some embodiments, the liposomes take the form of cationic liposomes, and the electrode structure is configured such that the anode side of the electrode structure is configured to transdermally deliver the composition including the liposomes, when current and/or a potential is applied to the electrode structure.

In some embodiments, the electrode structure includes at least a positive electrode and an antioxidant component holding portion capable of holding any of the disclosed compositions and/or formulations.

In some embodiments, the antioxidant component holding portion may be directly disposed on the front surface of the positive electrode and other components such as, for example, an ion exchange membrane, may be disposed between the positive electrode and the active ingredient holding portion insofar as the administration of liposomes by iontophoresis is not substantially hindered.

In some embodiments, the electrode structure comprises at least a positive electrode, an electrolyte holding portion for holding electrolyte disposed on the front surface of the positive electrode, an anion exchange membrane disposed on the front surface of the electrolyte holding portion, and an antioxidant component holding portion for holding any of the disclosed compositions and/or formulations. In some embodiments, a cation exchange membrane may be disposed on the front surface of the above-mentioned active ingredient holding portion.

As shown in FIG. 1, in some embodiments, an iontophoresis device 1 may include any of the disclosed electrode structures, or any other structure suitable for iontophoretic delivery of the active ingredient or any of the disclosed compositions and/or formulations. In some embodiments, the iontophoresis device 1 may include at least a power supply 2, an electrode structure 3 connected to the power supply 2, and a counter electrode structure 4. The electrode structure 3 may serve to hold any of the disclosed compositions and/or formulations. The structure of the counter electrode 4 is not limited insofar as the administration of liposomes by iontophoresis is not substantially hindered. For example, the counter electrode 4 may include a negative electrode 4, an electrolyte holding portion 42 for holding electrolyte disposed on the front surface of the negative electrode 4, and an ion exchange membrane disposed on the front surface of the electrolyte holding portion 42. The above-mentioned ion exchange membrane may be an anion exchange membrane or a cation exchange membrane, and preferable is an anion exchange membrane.

An example of an electrode structure 3 and an iontophoresis device 1 is illustrated in FIG. 1. Further examples include those disclosed in, for example, International Publication WO 03/037425 A1.

Liposomes may migrate to a side opposite to the positive electrode due to an electric field resulting from applying an electric current, and may be efficiently emitted from the electrode structure. In some embodiments, a method of operating an iontophoresis device, includes placing the electrode structure 3 comprising a plurality of liposomes carrying an active ingredient, and the counter electrode structure 4, on the skin surface of a living body 5, and applying a sufficient electric current to the iontophoresis device 1, so as to emit a substantial amount of the liposomes held in active ingredient holding 34 portion of the electrode structure.

In the above-mentioned iontophoresis device 1, the active ingredient holding portion 34 or the electrolyte holding 32 portion may be formed of a reservoir (electrode chamber) which is, for example, formed of acryl and is filled with any of the disclosed compositions and/or formulations, or with an electrolyte, and may be formed of a thin film body having properties of holding and/or retaining the disclosed compositions and/or formulations, or electrolyte. With respect to the thin film body, the same material can be used in the active ingredient holding portion 34 and the electrolyte holding portion 32.

The disclosed methods and device may employ any suitable electrolyte. In some embodiments, a suitable electrolyte can be selected based on the conditions and properties of the active ingredient. However, electrolytes that adversely affect the skin of a living body due to an electrode reaction should be avoided. Suitable electrolytes include organic acids and salts thereof. Those organic acids and salts thereof that take part or exist in a metabolic cycle of a living body are generally preferable from the viewpoint of non-toxicity. For example, suitable electrolytes include lactic acid and fumaric acid. In some embodiments, the suitable electrolyte is a one to one (1:1) aqueous solution of 1M lactic acid and 1M sodium fumarate.

It is important that the thin film body forming the active ingredient holding unit have the ability to absorb and/or retain any of the disclosed compositions, formulations, and/o electrolyte and to have the ability to migrate ionized liposomes absorbed in and/or retained by the thin film body under predetermined electric field conditions to the skin side (ion transportation ability, ion electrical conductivity). Exemplary materials having both favorable absorbance and retaining properties and favorable ion transportation ability include a hydrogel body of an acrylic resin (acrylic hydrogel membrane), a segmented polyurethane-based gel membrane, an ion conductive porous sheet for the formation of a gel-like solid electrolyte (e.g., a porous polymer which: is disclosed in Japanese Patent Application Laid-open No. Sho 11-273452; and is based on an acrylonitrile copolymer containing 50 mol % or more, or preferably 70 to 98 mol % or more of acrylonitrile and having a porosity of 20 to 80%), and the like. When adding (e.g., impregnating, permeating, loading, and the like) any of the disclosed compositions and/or formulations to the above-mentioned active ingredient holding unit 34 the impregnation and/or permeation degree (100×(WD)/D[%], where D represents a dry weight and W represents a weight after impregnation) is preferably from about 30% to about 40%.

The conditions for loading the antioxidant component holding portion 34 or the electrolyte solution holding portion 32 with any of the disclosed compositions, formulations, and/or electrolytes are suitably determined according to the amount of electrolyte or ionic drug to be loaded, the absorption rate, etc. In some embodiments, the loading of the antioxidant component holding portion is performed at, for example, 40° C. for 30 minutes.

In some embodiments, the inert electrode may be composed of, for example, a conductive material such as carbon or platinum and may be used as the electrode of the electrode assembly.

In some embodiments, a cation exchange membrane and an anion exchange membrane can be used in combination in the electrode assembly. Examples of cation exchange membranes include NEOSEPTA's manufactured by Tokuyama Soda, Co., Inc. (CM-1, CM-2, CMX, CMS, CMB, and CLE 04-2). Examples of anion exchange membranes include NEOSEPTA's manufactured by Tokuyama Soda, Co., Inc. (AM-1, AM-3, AMX, AHA, ACH, ACS, ALE 04-2, and AIP-21). Further examples of the membranes include a cation exchange membrane obtained by partially or entirely filling the pore portions of a porous film with an ion exchange resin having a cation exchange function and an anion exchange membrane obtained by partially or entirely filling the pore portions of a porous film with an ion exchange resin having an anion exchange function.

Details about the respective components and the like described above may be found in, for example, International Patent WO 03/037425A1 by the applicant of the present disclosure, the entire contents of which are incorporated into the present disclosure.

### EXAMPLE 2

### Preparation of Liposome Formulation

First, a liposome formulation for iontophoresis was prepared by encapsulating superoxide dismutase (SOD) (an active oxygen-extinguishing enzyme) in a liposome comprising a cationic lipid DOTAP with a stable lipid membrane composition capable of being used in iontophoresis by the following method.

250 μL of a solution of 10 mM of DOTAP (Avanti Polar Lipids, Inc.) in CHCl₃, 125 μL of a solution of 10 mM of cholesterol (hereinafter referred to as “Chol”; Avanti Polar Lipids, Inc.) in CHCl₃, and 250 μL of a solution of 10 mM of yolk phosphatidylcholine (NOF CORPORATION) in CHCl₃ were mixed, and 500 μL of CHCl₃ were added to the mixture, whereby a suspension (molar ratio; DOTAP: Chol:Rho-DOPE=7:3:0.1) was obtained. After removal of the solvent of the suspension by distillation, under reduced pressure with an evaporator, 400 μL of CHCl₃ were added to the remainder, and the solvent of the mixture was removed by distillation under reduced pressure again, whereby a lipid film was obtained. 1 mL of a 10-mM HEPES buffer and 0.5 mL of a solution of 5 mg (corresponding to 4,470 units/mg)/ml of SOD (manufactured by Sigma-Aldrich) in a 10-mM phosphate buffer (pH 7.4) were added to the lipid film. The resultant mixed liquid was left at room temperature for 10 minutes so as to be hydrated, and was then subjected to sonication (AU-25C ultrasonic cleaner manufactured by AIWA MEDICAL INDUSTRY CO., LTD., 85 W, room temperature, 1 minute). Further, the mixed liquid was treated with an extruder (product name: Mini-Extruder, manufactured by Avanti Polar Lipids, Inc.) by using PC membranes each having a pore size of 400 nm or 100 nm (product name: Nuclepre Track-Etch Membrane, manufactured by Whatman), whereby a liposome suspension was obtained. The resultant liposome formulation had an average particle diameter in the range of about 260 to 400 nm.

### EXAMPLE 3

### Transdermal Administration Test

The liposome formulation of Example 3 was transdermally administered to the shaved back of a rat via iontophoresis using the following protocol.

First, anesthesia (1 mL of Nembutal (50 mg/ml) per 1 kg of a body weight) was administered to each SD rat (male, 9 weeks old, manufactured by CLEA Japan, Inc.), and the hair on the back of each rat was shaved. Next, as shown in FIG. 1, an iontophoresis device 1 including a power supply 2, a working electrode assembly 3, and a counter electrode assembly 4 was placed on a biological surface, such as, for example exposed skin 5. 100 μL of the above liposome suspension was applied in advance to a surface where the exposed skin 5 and the working electrode assembly 3 contacted with each other.

The working electrode assembly 3, of iontophoresis device 1, include as previously disclosed: a positive electrode 31; an electrolyte solution holding portion 32 for holding 1 mL of an electrolyte solution (physiological saline), the electrolyte solution holding portion 32 being placed on the front surface of the positive electrode 31; an anion exchange membrane 33; and an antioxidant component holding portion 34 for holding 200 μL of the liposome suspension, the antioxidant component holding portion 34 being placed on the front surface of the anion exchange membrane 33.

The counter electrode assembly 4 included: a negative electrode 41; an electrolyte solution holding portion 42 for holding 1 mL of an electrolyte solution, the electrolyte solution holding portion 42 being placed on the front surface of the negative electrode 41; a cation exchange membrane 43; an electrolyte solution holding portion 44 for holding 800 μL of a physiological saline, the electrolyte solution holding portion 44 being placed on the front surface of the cation exchange membrane 43; and an anion exchange membrane 45 placed on the front surface of the electrolyte solution holding portion 44. In addition, ion exchange membranes stored in a physiological saline in advance were used as the above anion exchange membranes 33 and 45 (ALE 04-2 manufactured by Tokuyama Soda, Co., Inc.), and the cation exchange membrane 43 (CLE 04-2 manufactured by Tokuyama Soda, Co., Inc.).

Next, the liposome formulation was administered to a number of rats with the iontophoresis device 1 shown in FIG. 1 using a current of about 1.14 mA (0.45 mA/cm²) for about 1 hour.

1 hour after the administration of the liposome formulation, a dye (8-methoxypsoralen) capable of producing active oxygen by being irradiated with ultraviolet light was applied to each rat to which the liposome formulation had been administered. After that, each rat to which the liposome formulation had been administered was irradiated with ultraviolet light from a UVA (365 nm) lamp for 4 hours (34.5 J/cm²).

Forty-four (44) hours after the completion of the irradiation, the skin of each rat to which the liposome formulation had been administered was harvested, and the amount of a lipid peroxide (amount of malon dialdehyde MDA) in the skin was determined by a Thiobarbituric acid (TBA) method. Simultaneously with the determination, the skin was evaluated for oxidative damage by immunostaining with an antibody for detecting oxidative damage (an anti-MDA antibody (lipid oxidation), an anti-(hexanoyl)lysine antibody (protein oxidation), or anti-8-OH-deoxyguanosine (DNA oxidation)).

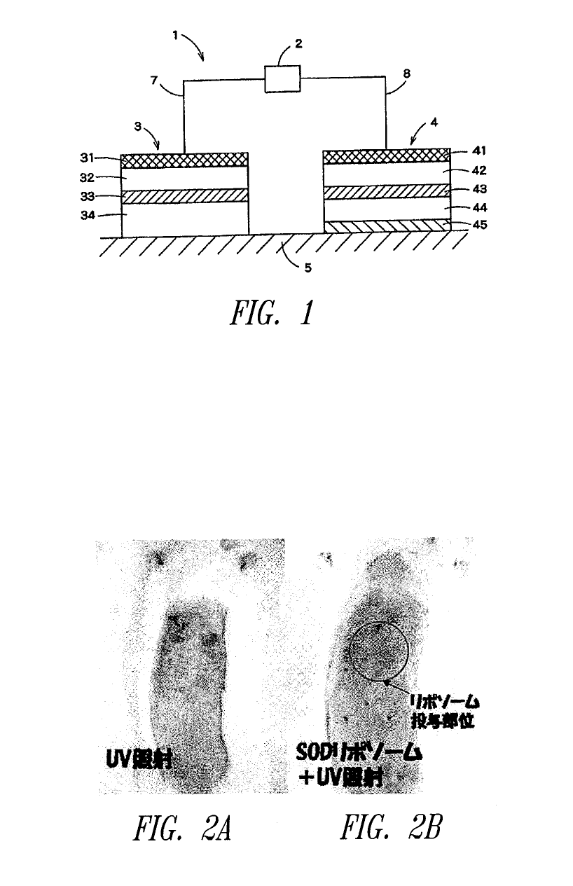

As a result, the skin of each rat irradiated with ultraviolet light exhibited signs of inflammation, and a spot was observed on the surface of the skin. However, the skin of each rat to which the SOD-carrying liposome formulation had been administered was identical to that before the irradiation with ultraviolet light (see FIGS. 2A and 2B). FIG. 2A shows the skin of a rat that was irradiated with ultraviolet light without the administration of the SOD-carrying liposome formulation, and the resulting inflammation on the skin. On the other hand, FIG. 2B shows the skin of a rat that was irradiated with ultraviolet light after the administration of the SOD-carrying liposome formulation, and that maintained the same state as that before the irradiation with ultraviolet light.

The skin of each rat irradiated with ultraviolet light was subjected to immunostaining. As a result, the skin was significantly stained by an antibody against oxidative damage, but the presence of oxidative damage was not observed in a rat to which the SOD-carrying liposome formulation had been administered. In addition, a comparison between the MDA amount of a rat to which the SOD-carrying liposome formulation had been iontophoretically administered and the MDA amount of a rat to which no liposome formulation had been iontophoretically administered showed that a value for the former was lower than a value for the latter.

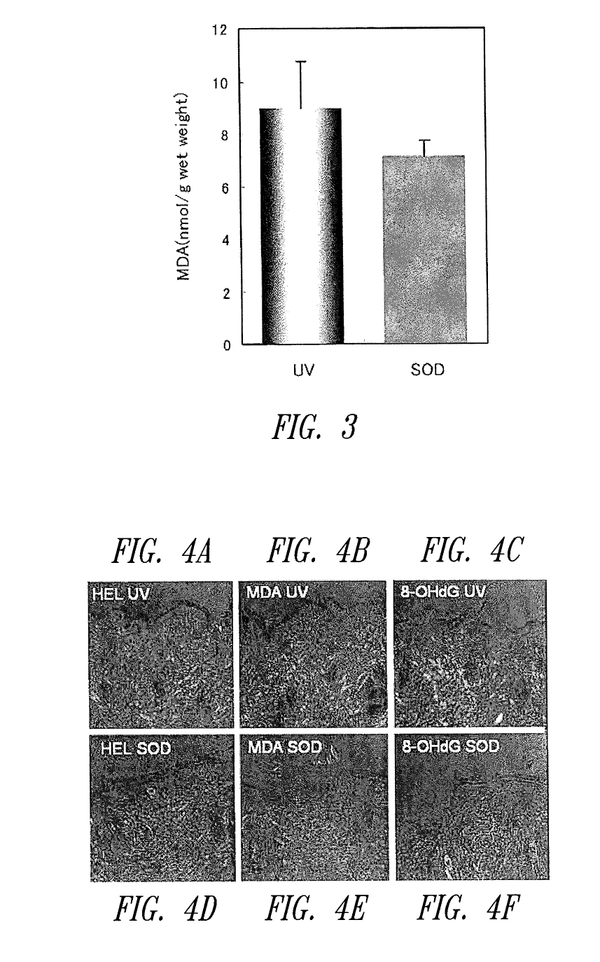

But the protecting effects on the skin against oxidative damage and the increase in MDA amount were not observed when the SOD-carrying liposome formulation was merely topically applied to the surface of the skin. FIGS. 3 and 4 show those results. The graph in FIG. 3 shows the result of the determination of the amount of a lipid peroxide (malon dialdehyde: MDA) of each of skin from a rat to which the SOD-carrying liposome formulation had not been iontophoretically administered (UV) and skin from a rat to which the SOD-carrying liposome formulation had been iontophoretically administered (SOD) after irradiation with UV.

On the other hand, FIGS. 4A to 4F each show photographs, obtained using a confocal laser microscope, of the observed fluorescence of skin sections of rats to which the SOD-carrying liposome formulation had not been iontophoretically administered (UV) and for rats to which the SOD-carrying liposome formulation had been iontophoretically administered (SOD) after irradiation with UV. The shown skin sections were subjected to immunostaining with each of various peroxidative damage marker antibodies.

FIG. 4A shows skin from a rat to which the SOD-carrying liposome formulation had not been administered (UV), and that was subjected to immunostaining with anti-(hexanoyl)lysine (HEL). FIG. 4B shows skin from a rat to which the SOD-carrying liposome formulation had not been administered (UV), and that was subjected to immunostaining with anti-malon dialdehyde (MDA). FIG. 4C shows skin from a rat to which the SOD-carrying liposome formulation had not been administered (UV), and that was subjected to immunostaining with anti-8-OH-deoxyguanosine (8-OHdG). FIG. 4D shows skin from a rat to which the SOD-carrying liposome formulation had been administered (SOD), and that was subjected to immunostaining with anti-(hexanoyl)lysine (HEL). FIG. 4E shows skin from a rat to which the SOD-carrying liposome formulation had been administered (SOD), and that was subjected to immunostaining with anti-malon dialdehyde (MDA). FIG. 4F shows skin from a rat to which the SOD-carrying liposome formulation had been administered (SOD), and that was subjected to immunostaining with anti-8-OH-deoxyguanosine (8-OHdG).

These results confirmed that the disclosed liposome compositions and/or formulations diffused from a pore to the inside of skin while maintaining its structure, and, further confirmed that the administration of an SOD-carrying liposome formulation to the inside of skin by iontophoresis was able to effectively treat (e.g., prevent, suppress, and the like) a dermatopathy in the skin due to ultraviolet light.

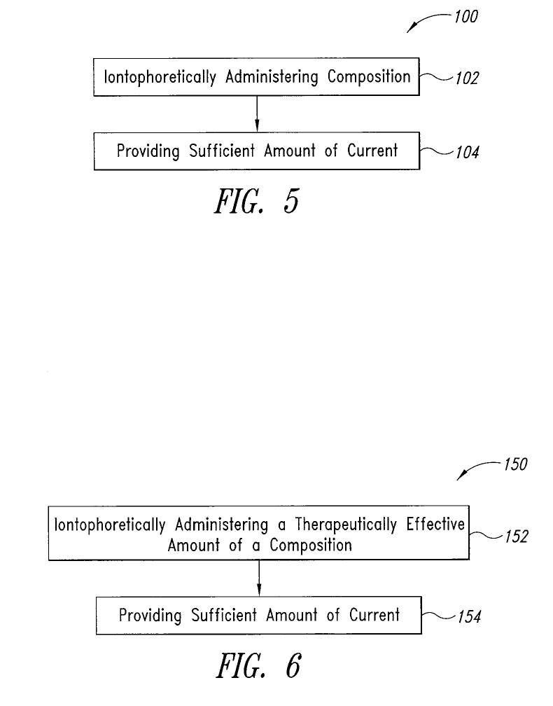

FIG. 5 shows an exemplary method 100 for treating a condition or a disease associated with oxidative stress in a living biological subject.

At 102, the method 100 includes iontophoretically administering to the living biological subject a composition comprising a plurality of liposomes comprising a cationic lipid, an amphiphilic glycerophospholipid having a saturated fatty acid moiety and an unsaturated fatty acid moiety, and one or more antioxidant enzymes selected from the group consisting of superoxide dismutase (SOD), glutathione peroxydase (GSH-Px), and catalase, the one or more antioxidant enzymes being carried by the plurality of liposomes. In some embodiments, the cationic lipid is present in a molar ratio of the cationic lipid to the amphiphilic glycerophospholipid of about 3:7 to about 7:3. In some embodiments, the liposome comprises an average particle diameter ranging from about 400 to about 1000 nm.

At 104, the method 100 includes providing a sufficient amount of current to deliver a therapeutic effective amount of the composition to the living biological subject. In some embodiments, providing the sufficient amount of current comprises providing a current ranging from about 0.1 mA/cm² to about 0.6 mA/cm².

In some embodiments, the condition associated with oxidative stress is an imbalance of reactive oxygen species. In some embodiments, providing the sufficient amount of current comprises providing an amount sufficient to iontophoretically administer an effective amount of the composition to a region in the living biological subject so as to lessen an imbalance of reactive oxygen species within the region.

In some embodiments, the disease associated with oxidative stress is a skin disease resulting from exposure to ultraviolet radiation. In some embodiments, the disease associated with oxidative stress is atherosclerosis, Parkinson's disease, Alzheimer's disease, skin cancers, skin tumor development, actinic keratosis, or malignant melanoma.

FIG. 6 shows an exemplary method 150 for preventing oxidative damage in a biological subject.

At 152, the method 150 includes iontophoretically administering to the biological subject in need of such treatment a therapeutically effective amount of a composition comprising a plurality of liposomes comprising a cationic lipid, an amphiphilic glycerophospholipid having a saturated fatty acid moiety and an unsaturated fatty acid moiety, and one or more antioxidants and/or antioxidant enzymes. In some embodiments, the one or more antioxidants and/or antioxidant enzymes are carried by the plurality of liposome.

In some embodiments, one or more antioxidants are selected from the group consisting of fat-soluble antioxidants such as, for example, α-tocopherol (vitamin E), β-carotene, astaxanthin, lycopene, capsaicin, water-soluble antioxidants such as, for example, ascorbic acid (vitamin C), polyphenol antioxidants such as curcumin, cysteine, and the like. In some embodiments, one or more antioxidant enzymes are selected from the group consisting of superoxide dismutase (SOD), glutathione peroxydase (GSH-Px), and catalase.

In some embodiments, the cationic lipid is present in a molar ratio of the cationic lipid to the amphiphilic glycerophospholipid of about 3:7 to about 7:3.

In some embodiments, iontophoretically administering to the biological subject in need of such treatment the therapeutically effective amount of a composition comprises providing a current ranging from about 0.1 mA/cm² to about 0.6 mA/cm² for a pre-selected period of time. In some embodiments, iontophoretically administering to the biological subject in need of such treatment the therapeutically effective amount of a composition comprises providing a current ranging from about 0.3 mA/cm² to about 0.5 mA/cm² for a pre-selected period of time. In some embodiments, iontophoretically administering to the biological subject in need of such treatment the therapeutically effective amount of a composition comprises providing a current of about 0.45 mA/cm² for a pre-selected period of time.

In some embodiments, iontophoretically administering to the biological subject in need of such treatment the therapeutically effective amount of a composition further comprises iontophoretically administering one or more antioxidant components selected from the group consisting of fat-soluble antioxidants, water-soluble antioxidants, and polyphenol antioxidants carried by the plurality of liposome.

At 154, the method 150 may further include providing a sufficient amount of current to deliver a therapeutic effective amount of the composition to the biological subject.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition for iontophoretic delivery of one or more antioxidant enzymes, comprising:
a plurality of liposomes comprising:
a cationic lipid, and
an amphiphilic glycerophospholipid having a saturated fatty acid moiety and an unsaturated fatty acid moiety; and
one or more antioxidant enzymes selected from the group consisting of superoxide dismutase (SOD), glutathione peroxydase (GSH-Px), and catalase, the one or more antioxidant enzymes being carried by the liposomes.

2. The composition according to claim 1, further comprising at least one antioxidant, the at least one antioxidant selected from the group consisting of fat-soluble antioxidants, water-soluble antioxidants, and polyphenol antioxidants.

3. The composition according to claim 1, further comprising at least one antioxidant, the at least one antioxidant selected from selected from the group consisting of α-tocopherol, β-carotene, astaxanthin, lycopene, capsaicin, ascorbic acid, and curcumin, cysteine.

4. The composition according to claim 1 wherein the cationic lipid comprises a C₁₋₂₀ alkane substituted with a C₁₋₂₂ acyloxy group and a triC₁₋₆ alkylammonium group.

5. The composition according to claim 1 wherein the cationic lipid comprises a C₁₋₂₀ alkane substituted with at least two C₁₋₂₂ acyloxy groups and at least one triC₁₋₆ alkylammonium group.

6. The composition according to claim 1 wherein the cationic lipid comprises 1,2-dioleoyloxy-3-(trimethylammonium)propane.

7. The composition according to claim 1 wherein the amphiphilic glycerophospholipid comprises phosphatidylcholine or an egg-yolk phosphatidylcholine.

8. The composition according to claim 1 wherein the saturated fatty acid moiety is a C₁₂₋₂₂ saturated fatty acid.

9. The composition according to claim 1 wherein the saturated fatty acid moiety is selected from the group consisting of palmitic acid, lauric acid, myristic acid, pentadecylic acid, margaric acid, stearic acid, tuberculostearic acid, arachidic acid, and behenic acid.

10. The composition according to claim 1 wherein the unsaturated fatty acid moiety comprises 1, 2, 3, 4, 5 or 6 carbon-carbon unsaturated double bonds.

11. The composition according to claim 1 wherein the unsaturated fatty acid moiety is C₁₄₋₂₂ unsaturated fatty acid.

12. The composition according to claim 1 wherein the unsaturated fatty acid moiety is selected from the group consisting of oleic acid, myristoleic acid, palmitoleic acid, elaidic acid, vaccenic acid, gadoleic acid, erucic acid, nervonic acid, linolic acid, α-linoleic acid, eleostearic acid, stearidonic acid, arachidonic acid, eicosapentaenoic acid, clupanodonic acid, and docosahexaenoic acid.

13. The composition according to claim 1 wherein a molar ratio of the cationic lipid to the amphiphilic glycerophospholipid is from about 3:7 to about 7:3.

14. The composition according to claim 1 wherein a molar ratio of the cationic lipid to the amphiphilic glycerophospholipid is from about 4:6 to about 6:4.

15. The composition according to claim 1, wherein the liposome further comprises a sterol, the sterol present in a molar ratio of the cationic lipid to the sterol of from about 3:7 to about 7:3.

16. The composition according to claim 15 wherein the sterol is selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether.

17. The composition according to claim 15 wherein the sterol is selected from the group consisting of cholesterol, cholesteryl lanolate, cholesteryl oleate, cholesteryl nonanate, cholesteryl macadaminate, and polyoxyethylene dihydrocholesteryl ether.

18. The composition according to claim 15 wherein the sterol is cholesterol.

19. The composition according to claim 15 wherein a molar ratio of the amphiphilic glycerophospholipid to the sterol is from about 3:7 to about 7:3.

20. The composition according to claim 15 wherein a molar ratio of the cationic lipid to the total of the amphiphilic glycerophospholipid and the sterol is from about 3:7 to about 7:3.

21. The composition according to claim 15 wherein a molar ratio of the cationic lipid, to the amphiphilic glycerophospholipid, and to the sterol is about 2:1:1.

22. The composition according to claim 1 wherein an average particle diameter of the liposome is about 400 nm or more.

23. The composition according to claim 1 wherein an average particle diameter of the liposome ranges from about 400 nm to about 1000 nm.

24. A method for treating a condition or a disease associated with oxidative stress in a living biological subject comprising:
iontophoretically administering to the living biological subject a composition comprising a plurality of liposomes comprising a cationic lipid, an amphiphilic glycerophospholipid having a saturated fatty acid moiety and an unsaturated fatty acid moiety, and one or more antioxidant enzymes selected from the group consisting of superoxide dismutase (SOD), glutathione peroxydase (GSH-Px), and catalase, the one or more antioxidant enzymes being carried by the plurality of liposomes, the cationic lipid present in a molar ratio of the cationic lipid to the amphiphilic glycerophospholipid of about 3:7 to about 7:3, and the liposome having an average particle diameter ranging from about 400 to about 1000 nm; and
providing a sufficient amount of current to deliver a therapeutic effective amount of the composition to the living biological subject.

25. The method of claim 24 wherein providing the sufficient amount of current comprises providing a current ranging from about 0.1 mA/cm² to about 0.6 mA/cm².

26. The method of claim 24 wherein providing the sufficient amount of current comprises providing sufficient current to iontophoretically administer an effective amount of the composition to a region in the living biological subject so as to lessen an imbalance of reactive oxygen species within the region.

27. The method of claim 24 wherein the condition associated with oxidative stress is an imbalance of reactive oxygen species.

28. The method of claim 24 wherein the diseases associated with oxidative stress is a skin disease resulting from exposure to ultraviolet radiation.

29. The method of claim 24 wherein the diseases associated with oxidative stress is atherosclerosis, parkinson's disease, Alzheimer's disease, skin cancers, skin tumor development, actinic keratosis, or malignant melanoma.

30. A method for preventing oxidative damage in a biological subject comprising:
iontophoretically administering to the biological subject in need of such treatment a therapeutically effective amount of a composition comprising a plurality of liposomes comprising a cationic lipid, an amphiphilic glycerophospholipid having a saturated fatty acid moiety and an unsaturated fatty acid moiety, and one or more antioxidant enzymes selected from the group consisting of superoxide dismutase (SOD), glutathione peroxydase (GSH-Px), and catalase, the cationic lipid present in a molar ratio of the cationic lipid to the amphiphilic glycerophospholipid of about 3:7 to about 7:3, and the one or more antioxidant enzymes being carried by the plurality of liposome.

31. The method of claim 30 wherein iontophoretically administering to the biological subject in need of such treatment the therapeutically effective amount of a composition comprises providing a current ranging from about 0.1 mA/cm² to about 0.6 mA/cm² for a pre-selected period of time.

32. The method of claim 30 wherein iontophoretically administering to the biological subject in need of such treatment the therapeutically effective amount of a composition comprises providing a current ranging from about 0.3 mA/cm² to about 0.5 mA/cm² for a pre-selected period of time.

33. The method of claim 30 wherein iontophoretically administering to the biological subject in need of such treatment the therapeutically effective amount of a composition comprises providing a current of about 0.45 mA/cm² for a pre-selected period of time.

34. The method of claim 30 wherein iontophoretically administering to the biological subject in need of such treatment the therapeutically effective amount of a composition further comprises iontophoretically administering one or more antioxidant selected from the group consisting of fat-soluble antioxidants, water-soluble antioxidants, and polyphenol antioxidants carried by the plurality of liposome.
